(19) United States
(12) Patent Application Publication
(10) Pub. No.: US 2003/0012804 A1
(43) Pub. Date: Jan. 16, 2003

(54) Aquacide and use

(75) Inventors: Stephen J. Cutler, Roswell, GA (US); Horace G. Cutler, Watkinsville, GA (US); David Wright, Solomon, MD (US); Rodger Dawson, Owings, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: AQUACIDE AND USE

(21) Appl. No.: 09/886,621

(22) Filed: Jun. 22, 2001

(51) Int. Cl. A61K 31/44; A01N 43/90; A01N 25/00; A01N 43/16; A01N 35/00; A01N 39/00

(52) U.S. Cl. 424/405; 424/613; 514/456; 514/680; 514/690; 514/306

(57) ABSTRACT

A method of controlling target aquatic microorganism pest populations by exposing the target population to an effective amount of an aquacidal compound. The aquacidal compounds are selected from the group consisting of quinones, anthraquinones, naphthalenediones, quinine, warfarin, coumarins, amphotalide, cyclohexadiene-1,4-dione, phenidione, pirdone, sodium rhodizonate, apirulosin and thymoquinone. The method is particularly effective for treating ballast water of ships or other enclosed volumes of water subject to transport between or among geographic areas to control the relocation of plants, toxic bacteria, and animals contained in the water.

### FIELD OF INVENTION

The present invention is directed to a method and compositions for controlling aquatic pests, including zoological organisms and plants. More specifically, the invention is directed to a method and composition for controlling, inhibiting, and terminating populations of aquatic and marine pest plants, organisms, and animals in a target treatment zone. The invention is particularly applicable for sterilizing a treated water volume (whether or not enclosed) of mollusks, dinoflagellates, bacteria and algae.

### BACKGROUND OF THE INVENTION

The discovery in the Summer of 1988 of the Eurasian zebra mussel Dressiness polymorph in the Great Lakes of North America represents one of the most significant events in the history of aquatic biological invasion. However, this was not the first event of a non-indigenous species entering into US water. Earlier, the spiny water flea Bythotrephes cedarstroemi and the ruffe Gymnocephalus cernuus had entered the United States from ballast water of European ports. It was soon discovered that zebra mussel had also entered the US via ballast water of European origin.

Since the summer of 1988, there have been a number of aquatic species that have entered into the United States via ballast water taken from ports of other countries. It is estimated that several hundred organisms have been introduced into the US via ballast water and/or other mechanisms, not limited to fisheries and ocean or coastal currents. As such, the integrity of the coastal waters of the United States and the Great Lakes basin has been substantially threatened by the increased rate of aquatic species introduction from other countries.

Prior to 1880, various methods for controlling ballast in ships were used. In fact, many streets in coastal towns are paved with stones once used for ship ballast. However, shortly before the turn of the century, water as ballast soon replaced these older methods of stabilizing ships. The rate of invasions by non-indigenous aquatic species rose dramatically since the turn of the century, with much of this being attributed to shipping. As transoceanic travel increased, so to has the inadvertent introduction of non-indigenous species that threaten natural waterways. This is a result of the diverse array of organisms that are able to survive the transoceanic travel in ship ballast water, sea chests, and on ship hulls. Of these, the ballast water of ships is one of the primary mechanisms by which organisms have invaded US waters.

Ballast water consists of either fresh or salt water that is pumped into a vessel to help control its maneuverability as well as trim, stability, and buoyancy. The water used for ballast may be taken at various points during the voyage including the port of departure or destination. Container ships may make as many as 12 port visits/ballast exchanges during a single round-the-world journey. Any planktonic species or larvae that is near the ballast intake may be taken up and transported to the next port of destination. Globally, an estimated 10 billion tons of ballast water are transferred each year. Each ship may carry from a few hundred gallons (about 2 metric tons) to greater than 100,000 metric tons depending on the size and purpose. More than 640 tons of ballast water arrive in the coastal waters of the United States every hour.

The risk of invasion through ballast water has risen dramatically in the past 20 years as a result of larger vessels being used to transport greater amounts of material into and out of the U.S. It is estimated that between 3000-10,000 species of plants and animals are transported daily around the world. In regard to those materials being brought into the U.S., it is of interest to note that materials which contain animals, fruits, vegetables, etc., must be inspected by the United States Department of Agriculture in order to satisfy requirements that potentially harmful non-indigenous species are excluded. The irony is that the ship may be able to release ballast water that has been contaminated with a non-indigenous species. It is through this mechanism that several hundred species have been introduced into the United States.

As noted above, one of the most notorious species introduced in the Great Lakes of North America is the Eurasian zebra mussel Dreissena polymorpha, which has become a major threat to inland water supplies from both a recreational and commercial aspect. Unfortunately, their range now extends from the Great Lakes to Louisiana and estimated economic losses are estimated at more than $4 billion for the calendar year 1999. This species is particularly prolific and a reproducing female can expel more than 40,000 fertile eggs per season which, upon hatching, may be found in colonies in excess of one hundred thousand per square meter. Furthermore, the colonies attach themselves to underwater structures that include, amongst others, water intake pipes, from which they can be readily disseminated into other environments, ship hulls, debris such as discarded automobile tires, sunken ships, and discarded metal drums. Established colonies often reach a thickness of 20 cm.

Of particular importance is the clogging of water intake pipes by zebra mussels that have a devastating industrial effect, especially in such uses as power plants, where there is a specific need for reliable water flow rates. Certain power plants have recorded a 50% water flow rate reduction following infestation and, in addition, zebra mussels appear to secrete substances, both in the living and dead state, that cause ferrous metal pipes to degrade. An associated problem also occurs in pipes that supply potable water because even following purification treatment, the water has an off flavor. This is attributed not only to the substances released by the living mussels, but especially by those that have died and are decaying. The latter most probably produce polyamines, such as cadaverine, which has a particularly obnoxious odor associated with decaying proteins and is most often noted in decaying meat.

Other detrimental environmental effects are the result of zebra mussel infestations both directly and indirectly. Of a direct nature are the effects on phytoplankton. Zebra mussels feed on phytoplankton, which are a source of food for fish, especially in lakes and ponds, thereby increasing the photosynthetic efficiency for other aquatic weed species because of increased clarity of the water. This has been shown to have dramatic effects on energy flow and food chains in some waters. Some fish species are threatened. The walleye, for example, thrives in cloudy water and it is generally believed by environmentalists that, increased water clarity resulted from zebra mussel activity will lead to the demise of that industry, presently estimated to be $900 million per year. Large-scale, multi-billion dollar degradations in native Great Lakes fisheries are already being felt as a result of competition from non-fishable species such as the Eurasian ruffe (Gymnocephalus cernuus) and the round goby (Proterorhinus marmoratus), which have been introduced through ballast water in the last two decades.

As a result of its feeding preferences, zebra mussels may radically alter the species composition of the algal community such that potentially harmful species may become abundant. An example is Microcystis, a blue-green alga of little nutritive value and capable of producing toxins which can cause gastrointestinal problems in humans. There are records of Microcystis blooms in Lake Erie and adjacent waterways. Toxic dinoflagellates such as Prorocentrum, Gymnodinium, Alexandrium and Gonyaulax often appear as blooms, sometimes known as “red tides”, in many parts of the world. Apart from causing serious (sometimes fatal) ailments in several vertebrate consumers, including humans, several of these organisms have had devastating effects on shellfish industries in several countries and it is now accepted that ballast-water introductions were responsible in many of these cases.

Reports of the introduction of the cholera bacterium, Vibrio cholera, to the Gulf coast of the United States have now been traced to the importation of this species associated with planktonic copepod (crustacean) vectors in ballast water arriving at Gulf coast ports from South America. This, in turn, had been transported from Europe to South American ports by similar means.

As a result of the introduction of non-indigenous species into the United States, and in order to reduce the possibility of the introduction of other organisms in the future, in 1990 the US Congress passed an act known as Public Law 101-646 “The Nonindigenous Aquatic Nuisance Prevention and Control Act” under the “National Ballast Water Control Program” which it mandates, among other things, studies in the control of the introduction of aquatic pests into the US. These control measures may include UV irradiation, filtration, altering water salinity, mechanical agitation, ultrasonic treatment, ozonation, thermal treatment, electrical treatment, oxygen deprivation, and chemical treatment as potential methods to control the introduction of aquatic pests. It is likely that other governments will pass similar legislation in the near future as the scope and costs of aquatic pest contamination become better understood.

Numerous methods and compositions have been proposed to control and inhibit the growth of various marine plants and animals. In particular, a number of compositions have been proposed to treat water and various surfaces having infestation of zebra mussels. Examples of various compositions are disclosed in U.S. Pat. Nos. 5,851,408, 5,160,047, 5,900,157 and 5,851,408. Treatment of various aquatic pests, other than toxic bacteria, is described in WO 00/56140 using juglone or analogs thereof.

These prior compositions and methods, although somewhat effective, have not been able to completely control the introduction of marine plants and animals into waterways. Accordingly, there is a continuing need in the industry for the improved control of aquatic pests in the form of plants and animals, preferably aquatic flora, fauna, and other organisms that can be suspended in water and are susceptible to geographic migration by water intake, currents, or tides. It would be particularly desirable to have an aquacidally effective composition that was effective against a broad spectrum of microorganisms at low concentrations with a short half life.

### SUMMARY OF THE INVENTION

It is an objective of the invention to provide a method and composition for treating water infested with a target aquatic pest to sterilize the treated water of the target aquatic pests.

An objective of the invention is to provide a method of treating water in a designated region of open water, an enclosed or a flow-restricted region to sterilize the area of aquatic pest microorganisms including plants, toxic bacteria, suspended animals, and other biologic organisms in sedimentary materials using at least one aquacidally active compound in an effective amount to be toxic to the target species and a peroxy compound in an amount sufficient to enhance the activity and/or spectrum of activity of the aquacidally active compound.

A further objective of the invention is to provide a method of treating ballast water in ships and intake pipes to control the transport of mollusks, dinoflagellates, toxic bacteria, algae and other microorganisms by sterilizing the ballast water with an aquacidally effective composition containing an aquacidal compound and a peroxy compound.

Still another object of the invention is to provide a method of treating a volume of water in an enclosed space or localized region of open water with a toxic amount of an aquacidally effective composition which is readily degraded to nontoxic by-products upon exposure to ultraviolet light.

Another object of the invention to provide a method of inhibiting the spread of translocatable aquatic pests such as adult zebra mussels, zebra mussel larvae, oyster larvae, algal phytoplankton Isochrysis galbana, Neochloris, chlorella, toxic dinoflagellates (e.g. Prorocentrum), marine and freshwater protozoans and toxic bacteria (including vegetative cultures and encysted forms thereof), adult and larval copepods (vectors of Vibrio Cholera and Vibrio fischeri) and other planktonic crustaceans, e.g., Artemia salina, fish larvae and eggs by treating the water with an amount of at least one aquacidally effective composition of the type described herein in a quantity and for a sufficient period of time to kill the target aquatic pests.

These and other objects of the invention that will become apparent from the description herein are attained by exposing a target population of aquatic pests in a designated body, stream, or water flowpath to a toxic amount of an aquacidal composition comprising: (a) at least one aquacidally effective compound and (b) a peroxy compound. Preferably, the aquacidally effective compound is selected from the following compounds, including their analogs and homologs: (i) quinones, (ii) anthraquinones, (iii) quinine, (iv) warfarin, (v) coumarins, (vi) amphotalide, (vii) cyclohexadiene-1,4-dione, (viii) phenidione, (ix) pirdone, (x) sodium rhodizonate, (xi) apirulosin, (xii) thymoquinone, and (xiii) naphthalenediones.

The aquacidal compositions according to the present invention with the peroxy compound are surprisingly more effective against many specific aquatic pest populations and are also effective against a broader spectrum of target pests than compositions without the peroxy component. When the aquacides of the invention allowed to remain in contact with the target pest organisms for a period within the range of several hours to several days, the target pest population is killed. The compounds are then degraded through the effects of ultraviolet light, oxidation, hydrolysis, and other natural mechanisms into benign by-products that allow the treated water to be returned to beneficial use.

### DETAILED DESCRIPTION OF THE INVENTION

The present invention is generally directed to a composition and its method of use for treating water that hosts a target population of aquatic pests with an aquacidally effective composition containing (a) an aquacidal compound and (b) a peroxy compound for a sufficient time to reduce the target population in the treated water to benign levels or sterilize the treated water of the target population. Thereafter, ambient ultraviolet radiation degrades the aquacidal compound into harmless by-products. Such an action at extremely low concentrations offers opportunities for control over translocatable aquatic pest organisms, control or sterilization of undesired microorganism “blooms” in geographically limited regions, and similar situations.

Aquacidal Composition

The aquacidal composition of the invention comprises: (a) at least aquacidally effective compound and (b) a peroxy compound in an amount of each that is sufficient to sterilize a volume of treated water from populations of target aquatic pest organisms after an extended contact time. Thereafter, the aquacide and peroxy compound degrade into benign by-products so that the water can be returned to beneficial use.

The aquacidally effective compound is selected from quinone, naphthalenedione, anthraquinone, and mixtures thereof. The quinones have the formula:

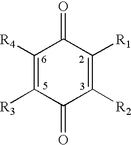

where

R₁ is hydrogen, methyl, hydroxy or methoxy group;

R₂ is hydrogen, hydroxy, methyl, methoxy or —NO₂ group;

R₃ is hydrogen, hydroxy, methyl or methoxy group; and

R₄ is hydrogen, methyl, methoxy, hydroxy, or —NO₂ group.

Examples of quinones found to be effective in controlling or inhibiting plant and animal growth in water include 1,4,benzoquinone (quinone), 2,5-dihydroxy 3,6-dinitro-p-benzoquinone (nitranilic acid), 2,6-dimethoxybenzoquinone, 3)-hydroxy-2-methoxy-5-methyl-p-benzoquinone (fumagatin), 2-methylbenzoquinone (toluquinone), tetrahydroxy-p-benzoquinone (tetraquinone), 2,3-methoxy-5-methyl-1,4-benzoquinone, 2,3-methoxy-5-methyl-1,4-benzoquinone, and mixtures thereof.

In further embodiments, the quinone can be an ubiquinone having the formula

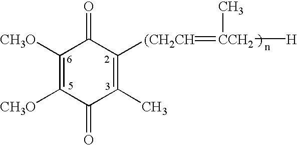

where n is an integer from 1 to 12. A particularly preferred ubiquinone has the formula above where n=10. In further embodiments, the ubiquinone has the above formula where n=6 to 10 and n is an integer.

In the embodiments where the marine plant and animal inhibiting composition is a naphthalenedione, such naphthalenediones have the formula:

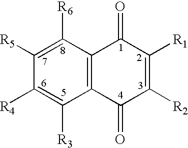

wherein:

R₁ is hydrogen, hydroxy or methyl group;

R₂ is hydrogen, methyl, sodium bisulfate, chloro, acetonyl, 3-methyl-2-butenyl or 2-oxypropyl group;

R₃ is hydroxy, hydrogen, methyl, chloro, methoxy, or 3-methyl-2-butenyl group;

R₄is hydrogen or methoxy group;

R₅ is hydrogen, hydroxy or methyl group;

R₆ is hydrogen or hydroxy group.

Examples of naphthalenediones include 1,4-naphthalenedione, 2-methyl-5-hydroxy-1,4-naphthalenedione (plumbagin), 2-methyl-1,4-naphthalenedione (Vitamin K₃), 2-methyl-2 sodium metabisulfite-1,4-naphthalenedione, 6,8-dihydroxy benzoquinone, 2,7-dimethyl-1-4-naphthalenedione (chimaphilia), 2,3-dichloro-1,4-naphthalenedione (dichlorine), 3-acetonyl-5,8-dihydroxy-6-methoxy-1,4-naphthalenedione (javanicin), 2-hydroxy-3-(3-methyl-2-butenyl)-1,4 naphthalenedione (lapachol), pirdone, juglone, and 2-hydroxy-3-methyl-1,4-naphthalenedione (phthiocol).

The anthraquinones have the formula:

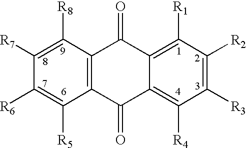

wherein

R₁ is hydrogen, hydroxy or chloro;

R₂ is hydrogen, methyl, chloro, hydroxy, carbonyl, or carboxyl group;

R₃ is hydrogen or methyl group;

R₄ is hydrogen;

R₅ is hydrogen or hydroxyl group;

R₆ and R₇ are hydrogen; and

R₈ is hydrogen or hydroxyl group.

Examples of anthraquinones that are suitable for treating water to control or inhibit marine plant and animal growth include 9,10 anthraquinone, 1,2-dihydroxyanthraquinone (alizarin), 3-methyl-1,8-dihydroxyanthraquinone, anthraquinone-2-carboxylic acid, 1-chloroanthraquinone, 2-methyl-anthraquinone, and 1-5 dihydroxyanthraquinone, 2-chloroanthraquinone.

Other biocdally effective compounds that can be used to control plant, animal, and microorganism growth either alone or in combination with each other and the quinones, naphthalenediones, and anthraquinones noted above include 9,10-dihydro-9-oxoanthracene (anthrone), 6′-methoxycinchonan-9-ol (quinine), 4-hydroxy-3-(3-oxo-1-phenyl butyl)-2H-1-benzopyran-2-one (warfarin), 2H-1-benzopyran-2-one (coumarin), 7-hydroxy-4-methylcoumarin, 4-hydroxy-6-methylcoumarin, 2[5-(4-aminophenoxy)pentyl]-1H isoindole 1,3-(2H)-dione (amphotalide), sodium rhdixonate, 2-phenyl-1,3-indandione (phenindione), 2,5 dihydroxy-3-undecyl-2,5 cyclohexadiene, spirulosin and thymoquinone.

Compounds that are particularly effective in controlling macroinvertebrates include 2,3-methoxy-5-methyl-1,4-benzoquinone, 2-methyl-1,4-naphthalenedione, 2-methyl-5-hydroxy-1,4-naphthalenedione, 2-methyl-2-sodium metabisulfite-1,4-naphthalenedione, 3-methyl-1,8-dihydroxyanthraquinone, 2-methyl-anthraquinone, 1,2-dihydroxyanthraquinone, 1,4-naphthalenedione, and mixtures thereof. These compounds are also effective in controlling the growth of dinoflagellates.

The amount of the aquacidal compound that is used will depend, in part, on the particular compound and the species of plant or animal being treated. As used herein, the term “effective amount”, “aquacidally effective”, and “aquacidal” refers to an amount that is able to kill the target species or render the target specie population inert and otherwise not viable of sustained vitality.

The amount of that aquacidal compound that is needed to treat water to kill a target plant or animal is an amount of less than about 1 wt %. Preferably, the aquacidal compound is added to the target body of water or water stream in an amount within the range of about 100 ppb to about 500 ppm (parts per million), more preferably in an amount within the range from about 500 ppb to about 300 ppm, most preferably within the range of 500 ppb to 250 ppm, and especially in an amount within the range of 1 ppm to about 250 ppm. Generally, the amount of the aquacidal composition used in treatment of ballast tank water will range from about 1 ppm to about 200 ppm.

The peroxy component of the present invention is characterized by the presence of the —O—O— peroxy group within the chemical structure. The peroxy compound can be added neat, in aqueous solution, or formed in-situ in an amount sufficient to enhance the effectiveness of the aquacidal compound or broaden the spectrum of target microorganisms against which the aquacidal compound is effective.

The peroxy compound can be added via a number of different compounds. Exemplary compounds useful as the peroxy compound include hydrogen peroxide and peroxyacid compounds such as t-butyl hydroperoxide, peroxy acetic acid, m-chloroperbenzoic acid, perbenzoic acid, performic acid, peroxycarboxylic acid, ester peracids, and mixtures thereof. An exemplary mixture comprises 40 to 60 wt. % carboxylic acid, 2 to 5 wt. % peroxycarboxylic acid and 0.1 to 3 wt. % hydrogen peroxide).

Suitable ester peroxyacids are characterized by the chemical structure:

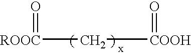

wherein:

x is from 1-4 carbon atoms, and

R is an alkyl group of 1-4 carbon atoms.

The amount of the peroxy compound that is used is within the range from about 0.001-100 wt % relative to the amount of the aquacidally effective compound. A preferred amount of peroxy compound is within the range from about 0.1-50 wt % and more preferably within the range of 1-25 wt % of the aquacidal compound.

The target pest population should be exposed to the aquacidal composition of the invention for a time sufficient to kill the target population. Adequate exposure periods for complete sterilization of target microorganisms are generally within the range of a at least one hour to a period of less than 96 hours (4 days) for both fresh water as well as salt water. A preferred exposure is within the range from about two hours to about 72 hours. Routine sampling and testing can be used to determine precise concentrations and exposure durations for a specific aquacidal compound, specific peroxy compound, peroxy:aquacide ratio, water type, target population, method of introduction, and temperature.

Water Bodies Suitable for Treatment

The water suitable for treatment with the present invention is one that is infested with a target microorganisms and can be located in a localized open water region, enclosed space or in a restricted flow path and can be any type of water that needs treatment by an environmentally acceptable sterilization process.

Exemplary bodies of water that can be treated according to the invention include ship ballast water reservoirs, commercial process water taken in from a static or dynamic body of water, water ready to be discharged into a holding reservoir or waterway, cooling or other forms of holding ponds, intakes ports or pipes, discharge ports or pipes, heat exchangers, sewage treatment systems, food and beverage processing plants, pulp and paper mills, power plant intake and outlet pipes, cooling canals, water softening plants, sewage effluent, evaporative condensers, air wash water, canary and food processing water, brewery pasteurizing water, “gray” water from various washing processes found onboard ships, and the like. It is envisioned that the aquacidal composition of the present invention can also be used to treat shore areas or swimming regions if an aquatic pest population has reduced the recreational value of a region of water in a localized or localizable area in an otherwise open body of water.

In its preferred embodiments, the aquacidal composition is added to ship ballast water at a concentration and for a period of exposure to the aquacidal compound that is effective in sterilizing the ballast water of target pests microorganisms. Such concentrations are typically sufficiently low to become diluted to a non-toxic level when discharged to a larger body of water so as to avoid or minimize harm to the indigenous species of plants and animals. Such a treatment method should help to prevent unintended migration of pest microorganisms between and among ports without significant capital expense or significant changes in commercial shipping practice.

The aquacidal composition of the invention is mixed into the target water as one homogeneous formulation or as discrete ingredient streams using standard dispensing devices and dispensing methods as known in the art. The composition can be dispensed as a single dose or over a period of time to maintain a desired concentration. Preferably, the aquacidal composition is introduced as a homogeneous mix at a turbulent zone or other area where agitation will mix the composition throughout the water to be treated. The composition can be fed intermittently, continuously, or in one batch.

Target Pest Populations

Aquatic pest organisms and populations that can be controlled, killed, or otherwise rendered benign by the method of the invention are generally not free ranging between geographical regions of their own efforts but are translocatable, i.e., they are subject primarily to the movement of the water currents or sediment around them. Such microorganisms are often captured in ballast water that is taken in at one port and discharged at another.

Aquatic pest microorganisms and populations that are targets for treatment according to the present invention include bacteria, viruses, protists, fungi, molds, aquatic pest plants, aquatic pest animals, parasites, pathogens, and symbionts of any of these organisms. A more specific list of aquatic pest organisms that can be treated according to the invention include, but are not limited to the following categories (which may overlap in some instances):

1) Holoplanktonic organisms such as phytoplankton (diatoms, dinoflagellates, blue-green algae, nanoplankton, and picoplankton) and zooplankton jellyfish, comb jellies, hydrozoan, polychaete worms, rotifers, planktonic gastropods, snails, copedods, isopods, mysids, krill, arrow worms, and pelagic tunicates), and fish.

2) Meroplanktonic Organisms such as Phytoplankton (propagules of benthic plants) and Zooplankton (larvae of benthic invertebrates such as sponges, sea anemones, corals, mollusks, mussels, clams, oysters, and scallops).

3) Demersal organisms such as small crustaceans.

4) Tychoplanktonic organisms such as flatworms, polychaetes, insect larvae, mites and nematodes.

5) Benthic organisms such as leaches, insect larvae and adults.

6) Floating, Detached Biota such as sea grass, sea weed, and marsh plants.

7) Fish and shellfish diseases, pathogens, and parasites.

8) Bythotrephes cederstroemi (spiny water flea, spiny tailed water flea).

9) Macroinvertebrates, such as mollusks, crustaceans, sponges, annelids, bryozoans and tunicates. Examples of mollusks that can be effectively controlled are mussels, such as zebra mussels, clams, including asiatic clams, oysters and snails.

In further embodiments, the animals being treated are selected from the group consisting of bacteria, e.g., Vibrio spp. (V. Cholera and V. Fischeri), Cyanobacteria (blue-green algae), protozoans, e.g. Crytosporidium, Giardia, Naeglaria, algae, e.g., Pyrrophyta (dinoflagellates, e.g. Gymnodinium, Alexandrium, Pfiesteria, Gonyaulax Glenodinium (including encysted forms)), Cryptophyta, Chrysophyta, Porifera (sponges), Platyhelminthes (flat-worms, e.g., Trematoda, Cestoda, Turbellaria), Pseudocoelomates (e.g., Rotifers, Nematodes), Annelid worms (e.g., polychaetes, oligochates), Mollusks (e.g., Gastropods, such as polmonate snails), Bivalves, e.g., Crassostrea (oysters), Mytilus (blue mussels), Dreissena (zebra mussels), Crustaceans, larval-adult forms of copepods, ostracods, mysids, gammarids, larval forms of decapods, and Larval teleost fish.

In one embodiment of the invention, mollusks, dinoflagellates, toxic bacteria, and algae are treated to inhibit growth by applying an effective amount of compound selected from the group consisting of 2,3-methoxy-5-methyl-1,4-benzoquinone, 2-methyl-1,4-naphthalenedione, and mixtures thereof.

One preferred embodiment of the invention is directed to a method of killing or inhibiting the growth of mollusks, dinoflagellates, toxic bacteria, and/or algae by exposing the mollusks, dinoflagellates, toxic bacteria, and/or algae to an effective amount of a quinone, anthraquinone, naphthalenedione, or mixture thereof. The method is effective in inhibiting the growth of toxic bacteria and mussels-particularly zebra mussels, and zebra mussel larvae, as well as other bivalves-by applying the aquacide compound to the water in an effective amount. In a preferred embodiment, mussels, and particularly zebra mussels and zebra mussel larvae, are treated to kill or inhibit their growth by exposing the zebra mussels to a toxic amount of a molluskocide compound selected from the group consisting of 2,3-methoxy-5-methyl-1,4-benzoquinone, 2-methyl-5-hydroxy-1,4-naphthalenedione, 2-methyl-1,4-naphthalenedione, 2-methyl-2-sodium metabisulfite-1,4-naphthalenedione, 3-methyl-1,8-dihydroxyanthraquinone, 2-methylanthraquinone, and mixtures thereof.

In a further embodiment, these aquacidal compositions can be incorporated into a solid or liquid bait for agricultural use to kill or inhibit the growth of snails and slugs. The bait can be a standard bait as known in the art. In other embodiments, the aquacidal composition is applied directly to the plant in an effective amount to treat the plant for controlling snails and slugs.

Coatings

The aquacidal compositions of the invention can also be added to paints and coatings with a suitable delivery mechanism to provide a sustained release of the aquacdal composition in a concentration sufficient to provide population control without adversely affecting the efficacy of the coating. The paint or coating composition can be applied to a surface, such as the hull of a boat, intake pipes, ship chests, anchors, and other underwater structures to prevent the plants and animals from growing and adhering to the surface.

The paint or coating composition can be conventional marine paint containing various polymers or polymer-forming components. Examples of suitable components including acrylic esters, such as ethyl acrylate and butyl acrylate, and methacrylic esters, such as methyl methacrylate and ethyl methacrylate. Other suitable components include 2-hydroxyethyl methacrylate and dimethylaminoethyl methacrylate that can be copolymerized with another vinyl monomer, such as styrene. The paint contains an effective amount of at least one aquacidal compound and an effective amount of the peroxy compound to inhibit plant an animal growth on a painted substrate.

As a paint or coating, the aquacidal composition is included in an amount to provide a concentration of the aquacidal compound at the surface of the coating of at least 500 ppb, preferably about 1 ppm to 50 wt %, and more preferably within the range of 100-500 ppm to provide a plant and animal controlling amount of the aquacide compound in the coating.

While various embodiments have been selected to illustrate the invention, it will be understood to those skilled in the art that various changes and modifications can be made to the process disclosed herein without departing from the spirit and scope of the invention as defined in the appended claims.

1. A method for controlling a population of target pest microorganisms by exposing said population to an effective amount of:
(a) at least one aquacidal compound selected from the group consisting of: (i) quinones, (ii) anthraquinones, (iii) quinine, (iv) warfarin, (v) coumarins, (vi) amphotalide, (vii) cyclohexadiene-1,4-dione, (viii) phenidione, (ix) pirdone, (x) sodium rhodizonate, (xi) apirulosin, (xiii) thymoquinone, and (xiii) naphthalenedione; and
(b) a peroxy compound.

3. The method of claim 1, wherein said population of target pest microorganisms is selected from the group consisting of viruses, protists, holoplanktonic organisms, and meroplanktonic organisms.

4. The method of claim 1 wherein said population of target pest organisms is selected from the group consisting of demersal organisms, benthic organisms, detached or floating biota, bacteria, encysted bacteria, and protozoans.

5. The method of claim 1 wherein said population of target pest organisms is comprises spiny water flea or bacteria.

6. A method according to claim 1 wherein said target aquatic pest is selected from the group consisting of bacteria, protozoans, algae, dinoflagellates, dinoflagellate cysts, zebra mussels, and zebra mussel larvae.

7. A method according to claim 1 wherein said target aquatic pest is a bacteria.

8. A method according to claim 7 wherein said bacteria is a Vibrio species.

9. A method according to claim 1 wherein said target organism is a dinoflagellate cyst.

10. The method of claim 1, wherein said aquacidal compound is a quinone having the formula: 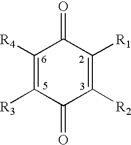
where
R₁ is hydrogen, methyl, hydroxy or methoxy group;
R₂ is hydrogen, hydroxy, methyl, methoxy or —NO₂ group;
R₃ is hydrogen, hydroxy, methyl or methoxy group; and
R₄ is hydrogen, methyl, methoxy, hydroxy, or —NO₂ group.

11. The method of claim 10, wherein said aquacidal compound is a naphthalenedione having the structural formula: 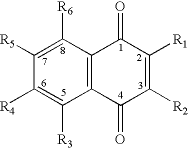
wherein:
R₁ is hydrogen, hydroxy or methyl;
R₂ is hydrogen, methyl, sodium bisulfate, chloro, acetonyl, 3-methyl-2-butenyl, or 2-oxypropyl;
R3 is hydrogen, methyl, chloro, hydroxy, methoxy or 3-methyl-2-butenyl;
R₄ is hydrogen or methoxy,
R₅ is hydrogen, hydroxy or methyl group;
R₆ is hydrogen or hydroxy group.

12. The method of claim 1, wherein said aquacidal compound is an anthroquinone having the formula: 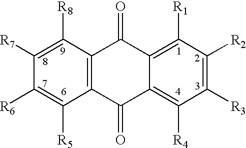
wherein
R₁ is hydrogen, hydroxy, chloro;
R₂ is hydrogen, methyl, chloro, hydroxy, carbonyl, or carboxyl group;
R₃ is hydrogen or methyl group;
R₄ is hydrogen;
R₅ is hydrogen or hydroxyl group;
R₆ and R₇ are hydrogen; and
R₈ is hydrogen or hydroxyl group.

13. The method of claim 1, wherein said aquacidal compound is an ubiquinone.

14. The method of claim 1, wherein said aquacidal compound is 2,3-methoxy-5-methyl-1,4-benzoquinone.

15. The method of claim 1, wherein said aquacidal compound is selected from the group consisting of 2-methyl-5-hydroxy-1,4-naphthalenedione, 2-methyl-1,4-naphthalenedione, 2-methyl-2-sodium metabisulfite-1,4-naphthalenedione, 3-methyl-1,8-dihydroxyanthraquinone, 2-methylanthraquinone, and mixtures thereof.

16. The method of claim 1, wherein said aquacidal compound is 2-methyl-1,4-naphthalenedione.

17. The method of claim 1 wherein said population of target pest organisms are located in a ballast water reservoir.

18. The method of claim 1 wherein said population of target pest organisms is Vibrio Cholera or Vibrio Fisheri.

19. The method of claim 1 wherein said peroxy compound is selected from the group consisting of hydrogen peroxide and peroxyacid compounds.

20. The method of claim 19 wherein said peroxy compound is selected from the group consisting of t-butyl hydroperoxide, peroxyacetic acid, m-chloroperbenzoic acid, perbenzoic acid, performic acid, peroxycarboxylic acid, ester peracids, and mixtures thereof.

21. The method of claim 19 wherein said peroxy compound comprises 40 to 60 wt. % carboxylic acid, 2 to 5 wt. % peroxycarboxylic acid and 0.1 to 3 wt. % hydrogen peroxide.

22. A composition useful for controlling a population of target pest microorganisms, said composition comprising effective amounts of:
(a) a peroxy compound, and
(b) at least one aquacidal compound selected from the group consisting of: (i) quinones, (ii) anthraquinones, (iii) quinine, (iv) warfarin, (v) coumarins, (vi) amphotalide, (vii) cyclohexadiene-1,4-dione, (viii) phenidione, (ix) pirdone, (x) sodium rhodizonate, (xi) apirulosin, (xiii) thymoquinone, and (xiii) naphthalenediones.

23. The composition of claim 22 wherein said peroxy compound includes hydrogen peroxide or a peroxyacid compound.

24. The composition of claim 23 wherein said peroxyacid compound is selected from the group consisting of t-butyl hydroperoxide, peroxyacetic acid, m-chloroperbenzoic acid, perbenzoic acid, performic acid, peroxycarboxylic acid, ester peracids, and mixtures thereof.

25. The composition of claim 23 comprising 40 to 60 wt. % carboxylic acid, 2 to 5 wt. % peroxycarboxylic acid and 0.1 to 3 wt. % hydrogen peroxide.

26. The composition of claim 22 wherein said peroxy compound is an ester peroxyacid that has the chemical structure: 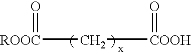
wherein:
x is from 1-4 carbon atoms, and
R is an alkyl group of 1-4 carbon atoms.

27. The composition of claim 22, wherein said aquacidal compound is a quinone having the formula: 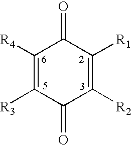
where
R₁ is hydrogen, methyl, hydroxy or methoxy group;
R₂ is hydrogen, hydroxy, methyl, methoxy or —NO₂ group;
R₃ is hydrogen, hydroxy, methyl or methoxy group; and
R₄ is hydrogen, methyl, methoxy, hydroxy, or —NO₂ group.

28. The composition of claim 23, wherein said compound is selected from the group consisting of 1,4-benzoquinone, 2,5-dihydroxy 3,6-dinitro p-benzoquinone, 2,6-dimethoxy benzoquinone, 3-hydroxy-2-methoxy-5-methyl-p-benzoquinone, 2-methylbenzo-quinone, tetrahydroxy-p-benzoquinone, 2,3-methoxy-5-methyl, 1-4-benzoquinone and mixtures thereof.

28. The composition of claim 22 wherein said aquacidal compound is a naphthalenedione having the formula: 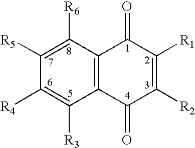
wherein:
R₁ is hydrogen, hydroxy, or methyl;
R₂ is hydrogen, methyl, sodium bisulfate, chloro, acetonyl, 3-methyl-2-butenyl, or 2-oxypropyl group;
R₃ is hydroxy, hydrogen, methyl, chloro, methoxy or 3-methyl-2-butenyl;
R₄ is hydrogen or methoxy;
R₅ is hydrogen, hydroxy or methyl; and
R₆ is hydrogen or hydroxy.

29. The composition of claim 28, wherein said aquacidal compound is selected from the group consisting of 1,4-naphthalenedione, 2-methyl-5-hydroxy-1,4-naphthalenedione, 2-methyl-1,4-naphthalenedione, 2-methyl-2 sodium metabisulfite-1,4-naphthalenedione, 6,8-dihydroxy benzoquinone, 2,7-dimethyl-1-4-naphalenedione, 2,3-dichloro-1,4-naphthalenedione, 3-acetonyl-5,8-dihydroxy-6-methoxy-1,4-naphthalenedione, 2-hydroxy-3-(3-methyl-2-butenyl)-1,4-naphthalenedione, pirdone, juglone, and 2-hydroxy-3-methyl-1,4-naphthalenedione.

30. The composition of claim 22, wherein said aquacidal compound is an anthroquinone having the formula: 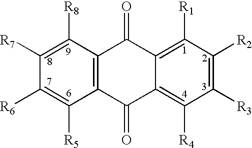
wherein
R₁ is hydrogen, hydroxy, chloro;
R₂ is hydrogen, methyl, chloro, hydroxy, carbonyl, or carboxyl group;
R₃ is hydrogen or methyl group;
R₄ is hydrogen;
R₅ is hydrogen or hydroxyl group;
R₆ and R₇ are hydrogen; and
R₈ is hydrogen or hydroxyl group.

31. The composition of claim 30, wherein said aquacidal compound is selected from the group consisting of 9,10 anthraquinone, 1,2-dihydroxyanthraquinone (alizarin), 3-methyl-1,8-dihydroxyanthraquinone, anthraquinone-2-carboxylic acid, 1-chloroanthraquinone, 2-methyl-anthraquinone, and 1-5 dihydroxyanthraquinone, 2-chloroanthraquinone.

32. The composition of claim 22, wherein said aquacidal compound is an ubiquinone.

33. The composition of claim 22, wherein said aquacidal compound is 2,3- methoxy-5-methyl-1,4-benzoquinone.

34. The composition of claim 22, wherein said aquacidal compound is selected from the group consisting of 2-methyl-5-hydroxy-1,4-naphthalenedione, 2-methyl-1,4-naphthalenedione, 2-methyl-2-sodium metabisulfite-1,4-naphthalenedione, 3-methyl-1,8-dihydroxyanthraquinone, 2-methylanthraquinone, and mixtures thereof.

35. The composition of claim 22, wherein said aquacidal compound is 2-methyl-1,4-naphthalenedione.

36. The composition of claim 22 wherein said peroxy compound is present within the range from about 0.001-100 wt % relative to said aquacidal compound.

37. The composition of claim 22 wherein said peroxy compound is present within the range from about 0.1-50 wt % relative to said aquacidal compound.

38. The composition of claim 22 wherein said peroxy compound is present within the range from about 1-25 wt % relative to said aquacidal compound.

39. A composition useful for killing a target population of mollusk pests in an aqueous system hosting said population, said composition comprising (a) a peracid, and (b) compound selected from the group consisting of 2-methyl-5-hydroxy-1 ,4-naphthoquinone, 2-methyl-1,4-naphthalenedione 2-methyl-2-sodium metabisulfate-1,4-naphthalenedione, 3-methyl-1,8-dihydroxyanthraquinone, 2-methylanthraquinone, and mixtures thereof.
